(19) United States
(12) Patent Application Publication
(10) Pub. No.: US 2025/0268932 A1
(43) Pub. Date: Aug. 28, 2025

(54) Oxygen-containing preparation for treating acne and wounds, a preparation method and an application device thereof

(71) Applicant: Yan ZHENG, Chengdu (CN)

(72) Inventor: Yan ZHENG, Chengdu (CN)

(21) Appl. No.: 19/208,544

(22) Filed: May 14, 2025

Related U.S. Application Data
(63) Continuation of application No. PCT/CN2023/123355, filed on Oct. 8, 2023.

(30) Foreign Application Priority Data
Nov. 16, 2022 (CN) 202211434066.2

(51) Int. Cl. A61K 33/00; A61F 13/00; A61K 9/70; A61P 17/10

(52) U.S. Cl. CPC A61K 33/00; A61F 13/00063; A61K 9/7053; A61P 17/10; A61F 2013/0037; A61F 2013/00646; A61F 2013/0071; A61F 2013/00872; A61F 2013/00906

(57) ABSTRACT

Oxygen-containing preparation for treating acne and wounds, a preparation method and an application device thereof. The oxygen-containing formulation comprises an oxygen bag that comprises a bag body configured for containing oxygen. A bag body wall of the bag body consists of a gas-permeable part configured for permeation of oxygen and controlling an oxygen transfer diffusion rate, and a gas-impermeable part. Alternatively, the bag body wall of the bag body consists of a gas-permeable part configured for permeation of oxygen and controlling the oxygen transfer diffusion rate. The thickness of the gas-permeable part is 5-500 μm, and the oxygen permeability of the gas-permeable part is 0.0000001 cm2·s·0.1 MPa-0.1 ml/cm2·s·0.1 MPa. The oxygen-containing formulation not only provides oxygen molecules for acne and the wound surface, thereby killing anaerobic bacteria and promoting wound healing, but provides oxygen partial pressure and oxygen pressure intensity, thereby ensuring the oxygen content during use.

### RELATED APPLICATIONS

The present patent document is a continuation of PCT Application Serial No. PCT/CN2023/123355, filed Oct. 8, 2023, designating the United States and published in English, which is hereby incorporated by reference.

The present patent document claims the benefit of priority to patent application No. 202211434066.2, filed Nov. 16, 2022, and entitled “Oxygen-containing preparation for treating acne and wounds, a preparation method and an application device thereof,” the entire contents of each of which are incorporated herein by reference.

### BACKGROUND

### 1. Technical Field

The invention relates to the technical field of oxygen-containing preparations, in particular to an oxygen-containing preparation for treating acne and wounds, a preparation method and an application device thereof.

### 2. Background Information

Acne common name acne, is a common disease of young men and women, frequently-occurring, according to literature reports, acne can be divided into several categories according to the severity of the condition, age stage, physical condition, etc. The most common is acne vulgaris, is also what we often say acne, acne vulgaris is the acne of young people, also commonly known as acne; In addition to acne vulgaris, there are conjunctivitis acne, which is a more complex type, manifested as serious nodules, cysts, sinuses and scars exist at the same time; Fulminant acne, which refers to a small number of patients with sudden exacerbation of the condition, and the appearance of fever, joint pain, anemia and other systemic symptoms; Medicated acne refers to acne-like skin lesions caused by androgens and glucocorticoids; In addition, there are infant acne, premenstrual acne, etc. More than 90% of adolescents can have the disease, and its good in the face, can also occur in the chest, back and other parts, its pathogenesis is complex, mainly due to the strong secretion of sebaceous glands, resulting in hair follicles, sebaceous duct keratosis embolism, in the anaerobic environment, parasitic in the hair follicle propionibacterium acne, a large number of proliferation, The formation of inflammatory erythema, papules, pustules, blackhead acne, nodules, cysts and other acne damage, severe can occur hyperplasia or concave atrophic scars, often affect beauty, social, work, marriage and quality of life, therefore, acne patients are very urgent for treatment.

At present, the clinical treatment of acne more drugs and some hormones, although in the treatment of acne has A unique effect, but at the same time the side effects of drugs are more, long-term use can appear such as redness peeling and endocrine disorders, as well as retinoic acid drugs on the skin irritation, teratogenicity.

Patent CN115737674A discloses a wound dressing based on gas oxygen supply and its production method. The dressing is mainly composed of a waterproof film, a gas storage container, an absorbent pad and a protective film from top to bottom. The container wall of the gas storage container is provided with a small hole, which is covered with a material that can be dissolved in water and/or a breathable film. The gas storage container stores oxygen with pressure. Although the method can provide oxygen independently, the method has the disadvantages that the amount of oxygen filling and release cannot be effectively controlled and the effect of oxygen release is poor, leading to poor treatment effect, and the problem of safety risk.

### BRIEF SUMMARY

Aiming at the shortcomings of the prior art, the invention provides an oxygen-containing preparation for treating acne and wounds, a preparation method and an application device thereof, which solves the problems that the oxygen release quantity cannot be effectively controlled in the prior art and the oxygen release effect is poor and there is safety risk.

On the one hand, the invention provides an oxygen-containing preparation for treating acne and wounds, comprising an oxygen bag. The oxygen bag comprises a bag body for holding oxygen, and the bag body wall of the bag body is composed of a breathable part for passing through oxygen and controlling the rate of oxygen transfer and diffusion;

Wherein, the thickness of the breathable part is 0.0000001 cm²·s·0.1 Mpa-0.1 ml/cm²·s·0.1 MPa. 5-500 μm, the oxygen permeability rate of the air permeability part is the oxygen bag by controlling the thickness of the air permeability part to adjust the oxygen transfer penetration speed. When the thickness of the breathable part is 5-500 μm, the oxygen release rate decreases from 0.1 ml/cm²·s·0.1 MPa to 0.0000001 cm²·s·0.1 MPa.

The “part” means that a part of the bag body is composed of a breathable part, and the remaining part is composed of an unbreathable part. “All” means that the bag body is all composed of a breathable part.

Further, the breathable part is a hydrophilic breathable film, and the hydrophilic breathable film comprises a non-porous breathable film or a microporous breathable film grafted with hydrophilic compounds.

The inventor finds that when the breathable part is a hydrophilic breathable film, the oxygen release rate is accelerated. This may be due to the high hydrophilicity and biocompatibility of the hydrophilic breathable film, the adhesion to skin cells is more firm, and it is easy to fuse with the extracellular matrix secreted by cells, which is conducive to the release of oxygen. In addition, it is conducive to the extracellular matrix and other liquids entering the oxygen bag, and the oxygen molecules are squeezed out of the oxygen bag, forming a certain oxygen partial pressure on the skin surface, and enhancing the therapeutic effect.

The non-porous breathable film transmits oxygen molecules from one side with high concentration and pressure to the other side through the transport-conduction-volatilization of the polymer carrier. The non-porous breathable film comprises a TPEE breathable film, a TPU breathable film and so on. The microporous permeable film makes oxygen effectively diffuse in a suitable channel by providing a suitable aperture for oxygen diffusion. The microporous breathable film comprises polytetrafluoroethylene film, polyethylene octene film, polypropylene film, polyether sulfone film, polyvinylidene fluoride film, polydimethylsiloxane film and so on.

Further, the hydrophilic compound comprises one or several kinds of polyvinyl alcohol, acrylic acid, polyethylene glycol and hydroxyethyl methacrylate.

The modification of hydrophilic compounds improved the surface structure and hydrophilicity of the microporous permeable film, made it easier to penetrate small oxygen molecules, and improved the permeability and biohistocompatibility of the microporous permeable film.

Further, the bag body is divided into a number of equally divided or unequally divided compartments independent of each other, each of which comprises a breathable part.

Wherein, the cross-sectional area of the compartment is 1-4 cm² and the volume is 1-8 cm³;

Or the compartment is a micro compartment, the size of the micro compartment is 0.01-100 μm, and the density is 10-5000/cm³.

The bag body of the invention can have different sizes and shapes according to needs, and the compartments also have different sizes and shapes, and each compartment is an independent individual and can be cut arbitrarily. For example, different shapes and sizes are designed in different parts of the face treatment to provide better adhesive feeling and treatment effect. When used for the treatment of sinus wounds and anaerobes in the cavity body shape, the micro-compartment is designed to ensure the release of oxygen and the therapeutic effect.

Further, the outer surface of the breathable part is covered with a gel layer, which comprises an oxygen carrier.

The gel layer has a certain adhesive property, which can not only carry oxygen, so that the oxygen molecules transferred from the air bag will not spread outside the area covered by the air bag, but also increase the oxygen concentration on the skin surface, and play the role of fixing the oxygen bag on the surface of acne and wound.

Further, the oxygen carrier includes one or several of perfluoro-n-butylfuran, perfluoro-tributylamine, fluororen E4, perfluoronaphthane, perfluoromethylnaphthane, perfluoro-tripropylamine, polyvinylpyrrolidone.

The oxygen carrier can not only carry oxygen to the surface of the skin, but also penetrate deep into the skin, so that the oxygen penetrates the skin into the interior of the tissue, thus enhancing the therapeutic effect of oxygen. In addition, it can also release cell carbon dioxide, so that the therapeutic effect is further enhanced. At the same time, it can add moisturizing small molecules in the oxygen carrier, such as hyaluronic acid.

On the other hand, the invention provides a preparation method of oxygen-containing preparations for treating acne and wounds, including the following steps:
S1: Preparation of a permeable part: preparation of a non-porous permeable film or hydrophilic modification of a micro-porous permeable film by surface grafting method to obtain a permeable part:
S2: Prepare oxygen bag: process the permeable part into oxygen bag by blister method, blow molding method or foaming method;
Or prepare the breathable part by blister, blow molding or foaming method, and hot press or glue the breathable part and the breathable part to get the oxygen bag;
Preferably, the preparation of a gel layer is included; Coating the gel layer on the surface of the breathable film of the oxygen bag.

On the other hand, the invention provides an application device of oxygen-containing preparations for treating acne and wounds, comprising an airtight container, an oxygen generator and a sterile packaging bag.

The breathable container is used for storing and providing oxygen to the oxygen bag. The breathable container comprises a cylinder body and a cover body.

The cylinder body and the cover body are sealed connected, and the cylinder body or the cover body is provided with an oxygen input port;

The oxygen generator is used to produce oxygen and deliver oxygen to an airtight container. The oxygen generator is communicated with the airtight container through the oxygen input port;

The aseptic packaging bag is used to pack the oxygen bag and keep the oxygen bag in a sterile state.

Further, the aseptic bag comprises a packaging bag body, a hot pressure sealing line and a tear Angle; Four sides of the packaging bag body are arranged with a hot pressure sealing line fixed connection with it.

On the other hand, the invention provides an oxygen-containing preparation for treating acne and wounds, which is used in preparing drugs for treating wounds or for reducing inflammatory response.

It can be understood that the hyperabsorbent resin particles such as sodium polyacrylate polymer can be added to the compartment during the preparation of the oxygen bag, which can absorb the wound seepage, avoid the wound immersion, prolong the service time, and at the same time extrude the gas molecules from the bubble to improve the treatment effect.

It is understood that when used, the wound can be covered with alginate sheet, hydrocolloid, oil gauze and other materials, and then the oxygen bag is affixed to the fixed, or the gas molecule permeable film surface coated with breathable adhesive material for fixing, you can also use gas molecule impermeable tape fixed.

The technical principle of the invention is as follows: The invention controls the transfer rate of oxygen molecules by the thickness of the air permeation part and the characteristics of the air permeation part of different materials, and the surface of the air permeation part is covered with a layer of gel, which has a certain adhesive property, can not only carry oxygen, so that the oxygen molecules transferred from the air bag will not spread to the area covered by the air bag, but also can increase the oxygen concentration on the skin surface. In addition, it can be applied on acne and wound surface to play the role of fixing oxygen bag. Further, the inventor also found that when the perfluoronaphthalene small molecule polymer is used as an oxygen carrier, it can not only carry oxygen to the surface of the skin, but also penetrate into the deep skin, so that the oxygen penetrates the skin into the internal tissue, thus enhancing the therapeutic effect of oxygen. And can also release cell carbon dioxide, so that the therapeutic effect is further enhanced.

Compared with the prior art, the invention has the following beneficial effects:
(1) The oxygen-containing preparation of the invention not only provides oxygen molecules for acne and wound, kills anaerobic bacteria and promotes wound healing; Moreover, the oxygen content in the process of use is guaranteed; At the same time, due to the way of gas exchange, carbon dioxide can be exchanged out of the cell to improve the therapeutic effect.
(2) The gel layer containing the oxygen carrier of the invention has a certain viscosity, which ensures that the sealing of the coating surface does not leak, while increasing the oxygen partial pressure and oxygen pressure of the contact surface. At the same time, the small molecule technology used can penetrate the oxygen molecule into the deep layer of the contact surface by the oxygen carrying material, so as to achieve the best therapeutic effect.
(3) The breathable part of the oxygen-containing preparation of the invention is a hydrophilic breathable film, which not only promotes the release of oxygen, but also has a synergistic effect with the gel layer, and jointly improves the therapeutic effect of the oxygen-containing preparation.
(4) The oxygen-containing preparation of the invention is a sterile product and can be sterilized by a variety of sterilization methods; It can realize sterilization-transportation-oxygenation of oxygen bag beside the bed-use, and ensure the safety and effectiveness of the product, especially the safety of sterile product transportation.
(5) The preparation process of the invention is simple and convenient.

### BRIEF DESCRIPTION OF THE DRAWINGS

FIG. 1 is a schematic diagram of the application device in embodiment 3 of the invention.

FIG. 2 is a side view of the oxygen-containing preparation in embodiment 3 of the invention.

FIG. 3 is the oxygen release curve of the oxygen-containing preparation in test example 1 of the invention.

In the above drawings: 1. Airtight container; 2. Oxygen generator; 3, aseptic packaging bag; 4, oxygen bag; 5, compartments; 6. Ventilation department.

### DETAILED DESCRIPTION OF THE DRAWINGS AND THE PRESENTLY PREFERRED EMBODIMENTS

The technical scheme of the invention is further explained in combination with the attached drawings and embodiments. GT700 water-based gel, tetrahydroxypropyl ethylenediamine and mevalonolactone are all purchased from Adicco.

### Embodiment 1 Preparation of Oxygenated Preparations for the Treatment of Acne and Wounds

1. Preparation of a microporous breathable film grafted with hydrophilic compounds

The polytetrafluoroethylene film and polyethylene octene film composite soaked in polyethylene glycol solution, soaking time 15 h, and then take out for drying treatment, that is, the hydrophilic microporous permeable film.
2. By blister method, the breathable part prepared by LDPE material is vacuumed and hot-pressed together with the hydrophilic microporous breathable film prepared in step 1 to form an oxygen bag with a thickness of 70 μm.
3. The gel made of perfluoronaphthylene was covered on the surface of the hydrophilic microporous permeable film; Or apply the gel layer directly to the skin surface.

The specific preparation of the gel layer is as follows:
Phase A: propylene glycol 5%, diglycerol 5%, PG carbonate 10%, digPEG18 methyl ether dimethylsilane 3%;
Phase B: deionized water 68.6+5, GT700 water-based gel 1.5%, EDTA0.2%, Arabic gum and xanthan gum 0.3%;
Phase C: tetrahydroxypropyl ethylenediamine 3% (PH6-7.5);
Phase D: mevalonolactone 3%, perfluoronaphthalane 5%, carbon dioxide and nitrogen molecular sieve adsorption micro-powder;
Phase A, phase B and phase D were homogenized and mixed at high speed, and 5% NaOH aqueous solution was added to adjust the PH value to 6-7 during the stirring process; Then phase A was added to phase B homogenized and mixed at high speed, phase C was added to continue homogenized and mixed at high speed, and finally phase D homogenized and mixed at high speed, and the canned was sealed.

The material of impermeable part also includes PET, PU, PA, PVC and so on.

### Embodiment 2 Preparation of Oxygenated Preparations for the Treatment of Acne and Wounds

1. Preparation of a microporous breathable film grafted with hydrophilic compounds

The polyvinylidene fluoride film is soaked in polyethylene glycol solution for 20h, and then taken out for drying treatment to obtain a hydrophilic microporous breathable film.
2. The hydrophilic microporous permeable film prepared in step 1 is processed into an oxygen bag with a thickness of 200 μm by blister method.
3. Make polyvinylpyrrolidone into a gel and cover the surface of the hydrophilic microporous permeable film.

### Embodiment 3 Application of Oxygenated Preparations for the Treatment of Acne and Wounds

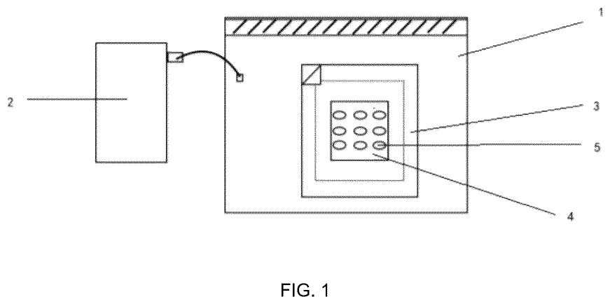

As shown in FIG. 1, the application device comprises an airtight container 1, an oxygen generator 2, and a sterile packaging bag 3. The air-tight container 1 is used for storage to provide oxygen to the oxygen bag 4. The air-tight container 1 comprises a cylinder and a cover body, the cylinder and the cover body are sealed connected, and the cylinder or cover body is provided with an oxygen input port;

The T oxygen generator 2 is used to generate oxygen and deliver oxygen to the airtight container 1. The oxygen generator 2 is communicated with the airtight container 1 through the oxygen input port;

The aseptic packaging bag 3 is used to pack the oxygen bag 4 and make the oxygen bag 4 in a sterile state, and the aseptic packaging bag 3 is arranged in the airtight container 1. The aseptic bag 3 comprises a packaging bag body, a hot pressure sealing line and a tear Angle; Four sides of the packaging bag body are arranged with a hot pressure sealing line fixed connection with it.

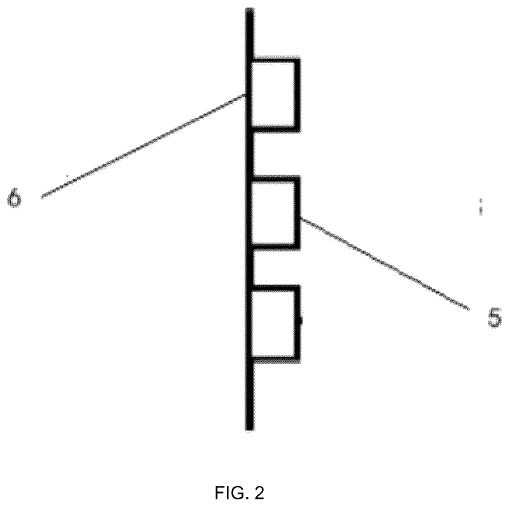

Prepared according to the method of embodiment 1, as shown in FIG. 1, oxygen bag 4 contains 9 compartments 5, each compartments 5 are independent of each other, the cross-sectional area of compartment 5 is 1 cm², the volume is 1 ml, and the air is inside compartment 5. As shown in FIG. 2, the bottom of compartment 5 is the ventilation section 6, and the oxygen permeability of the ventilation section 6 is 1.7 ml/cm²·h·0.1 MPa. The oxygen bag 4 was encapsulated in a sterile bag 3 and sterilized with ethylene oxide with a shelf life of 2 years.

When used, sterilized oxygen bag 4 with aseptic bag 3 is put into airtight container 1 for sealing 1 hour in advance, and oxygen is injected into airtight container 1 through oxygen generator 2. There is partial pressure difference between the airtight container 1 and various gas molecules of oxygen bag 4. After one hour, the partial pressure difference transfers oxygen molecules to oxygen bag 4. Take out the oxygen bag 4 with aseptic packaging bag 3 from the non-breathable container 1, open the aseptic packaging bag 3, and apply the ventilation part 6 side of the oxygen bag 4 on the acne or wound surface.

### Example 1 Breathable Effect Test

### (1) Skin Preparation:

a. Healthy Wistar rats were selected and given 10% chloral hydrate solution with intraperitoneal injection for anesthesia. The back hair was carefully removed with surgical scissors, and then the skin was carefully peeled off to remove subcutaneous fat and tissue;
b. The skin was rinsed with normal saline repeatedly, and small pieces of suitable size were cut and stored in a −20° C. refrigerator for use within one week;
c. Before the penetration experiment, remove the skin from the refrigerator and thaw it naturally in normal saline at room temperature;

### (2) Penetration Test:

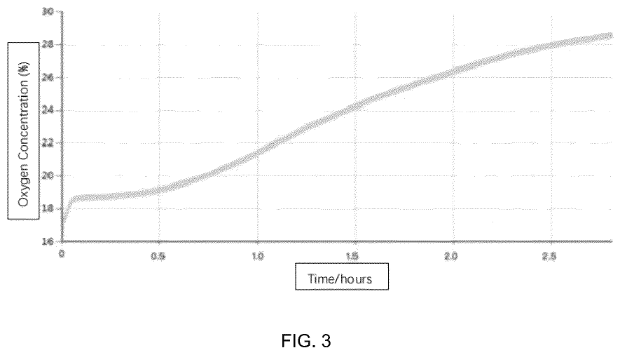

Using the mouse skin prepared in step (1) as the carrier, it was fixed on the side of the supply chamber of the micro-oxygen measurement equipment between the supply chamber and the receiving chamber. The oxygen bag prepared by Embodiment 1 was affixed to the skin of the mouse on the side of the supply chamber coated with gel layer. The skin of the mouse on the receiving chamber side was soaked in deionized water, placed on the vibrating screen, and placed in a constant temperature box. The temperature was set at 37 degrees Celsius, and the receiving chamber was connected with the sensor of a micro-oxygen tester; The oxygen content of the oxygen bag in this embodiment is 50 ml, and the volume of one side of the receiving chamber is 400 ml, as shown in the oxygen release curve diagram in FIG. 3.

Test Case 2 Oxygen-containing preparations are used to treat acne
Experimental Group 1: Oxygenated preparations prepared in Embodiment 3
Experimental group 2: Similar to experimental group, except that the microporous breathable film has not been hydrophilic modified.
Experimental group 3: similar to the experimental group, except that no gel layer is added.
Control group 1: Vitamin A cream.
Control group 2: Leave untreated.

Animals: 30 SD rats, male, all 3-5 weeks old and weighing about 100-120 g.

Preparation of rat acne model: The hair on the right ear of the rats was shaved and randomly divided into two groups: A group with 3 rats and B group with 27 rats. Group A: blank control group, group B: acne model group. Injections were started one week after normal feeding. Group A was not given any treatment; Group B was given Propionibacterium acnes suspension 50 μl in the middle of the inner auricle of the right ear, intradermal injection, once a day. After 7 days of injection, 2 rats were randomly selected and 4 ear pieces in the inner pinna of the right ear were prepared with a 3 mm diameter perforator. Routine HE pathological examination showed that acne-like lesions such as acne, papules and pustules appeared on the skin of rats in group B, which were red in color and hard in quality, indicating that a rat acne model had been formed.

Model grouping and results criteria: The remaining 25 rats were divided into 5 groups with 5 rats in each group. Groups 1-4 were treated with oxygenated preparations of experimental group 1, experimental group 2, experimental group 3 and control group 1, respectively, and group 5 was control group 2. They were respectively used in the center of the inner pinna of the right ear of rats. The dosage of control group 1 was 0.1 g. The dosage of experimental group 1-3 was one compartment. All groups were given the drug twice a day for 3 weeks. 24 h after the last administration, ear slice specimens (full skin layer) were prepared with a 3 mm perforator, and routine HE pathological examination was performed.

Results: After 3 weeks of subgroup administration, the number of spinal cell layers in experimental group 1 had returned to the normal 2 layers in the right ear without modeling, the hair follicles were free of keratinides, and the inflammatory cells in the dermis of local skin lesions were significantly reduced. Experimental group 2: the number of spinous cell layers recovered from the original 10 layers to 4 layers, the hair follicle keratins were small, and the local dermis inflammatory cells were reduced; Experimental group 3: The number of spinous cell layers recovered from the original 10 layers to 5 layers, the hair follicles had a moderate amount of keratins and were relatively loose, and the inflammatory cells in the dermis of the local skin lesions were reduced; In control group 1, the number of spinous cell layers was restored to 4 layers, the hair follicle had a small amount of keratins, and the local dermis inflammatory cells were reduced; In control group 2, the epidermis was still thickened, the hair follicles had a large amount of keratins, and the local dermis had a large number of inflammatory cells.

Finally, the above embodiments are used only to illustrate the technical scheme of the invention and not to limit it. Although the invention is described in detail with reference to the better embodiments, it should be understood by persons of ordinary skill in the art that the technical scheme of the invention may be modified or equivalent replaced without deviating from the object and scope of the technical scheme of the invention. They shall all be covered within the scope of the claims of the invention.

1. An oxygen-containing preparation for treating acne and wounds is characterized in that it comprises an oxygen bag, the oxygen bag comprises a bag body for holding oxygen, and the bag body wall of the bag body is partially or entirely composed of a breathable part for passing through oxygen and controlling the rate of oxygen transfer and diffusion;
Wherein, the thickness of the air permeability part is 5-500 μm, and the oxygen permeability rate of the air permeability part is 0.0000001 cm²·s·0.1 Mpa-0.1 ml/cm²·s·0.1 MPa.

2. As described in claim 1, an oxygen-containing preparation for the treatment of acne and wounds is characterized in that the breathable part is a hydrophilic breathable film, and the hydrophilic breathable film includes a non-porous breathable film or a microporous breathable film grafted by hydrophilic compounds.

3. An oxygen-containing preparation for the treatment of acne and wounds as described in claim 2 is characterized in that the hydrophilic compounds include one or several kinds of polyvinyl alcohol, acrylic acid, polyethylene glycol and hydroxyethyl methacrylate.

4. An oxygenated preparation for the treatment of acne and wounds, as described in claim 1, is characterized in that the bag body is separated into a number of equal or unequal compartments independent of each other, each of which includes a breathable part, wherein the cross-sectional area of the compartment is 1-4 cm² and the volume is 1-8 cm³; Or the compartment is a micro compartment, the size of the micro compartment is 0.01-100 μm, and the density is 10-5000/cm³.

5. An oxygen-containing preparation for the treatment of acne and wounds as described in claim 1 is characterized in that the outer surface of the breathable part is covered with a gel layer, and the gel layer includes an oxygen carrier.

6. An oxygen-containing preparation for the treatment of acne and wounds as described in claim 5 is characterized in that the oxygen-carrying carrier comprises one or more of perfluoro-n-butylfuran, perfluoro-Tbutylamine, fluororen E4, perfluoronaphthane, perfluoromethylnaphthane, perfluoro-tripropylamine, polyvinylpyrrolidone.

7. A method for the preparation of an oxygenated preparation for the treatment of acne and wounds described in any of the claim 1 is characterized in that it comprises the following steps:
S1: Preparation of a permeable part: preparation of a non-porous permeable film or hydrophilic modification of a micro-porous permeable film by surface grafting to obtain a permeable part:
S2: Prepare oxygen bag: process the permeable part into oxygen bag by blister method, blow molding method or foaming method;
Or by blister, blow molding or foaming method to prepare the breathable part, the breathable part and the breathable part by hot pressing or rubber composite oxygen bag;
Preferably, the preparation of a gel layer is included; Covering the surface of the breathable part of the oxygen bag with the gel layer.

8. The application device of oxygen-containing preparation for the treatment of acne and wound is characterized in that: including an airtight container, an oxygen generator and a sterile bag, the airtight container is used to store and provide oxygen to the oxygen bag, the airtight container comprises a cylinder and a cover body, the cylinder and the cover body sealing connection, the cylinder or cover body is provided with an oxygen input port;
The oxygen generator is used to produce oxygen and deliver oxygen to the impermeable container, and the oxygen generator is communicated with the impermeable container through the oxygen input port;
The aseptic packaging bag is used to pack the oxygen bag and keep the oxygen bag in a sterile state.

9. As described in claim 8, an application device of oxygen-containing preparations for the treatment of acne and wounds is characterized in that the aseptic packaging bag comprises a packaging bag body, a hot pressure sealing line and a tear Angle, and the four sides of the packaging bag body are arranged with a hot pressure sealing line fixed connection with it.

10. The application of any of the oxygen-containing preparations for the treatment of acne and wounds mentioned in claim 1 in the preparation of drugs for the treatment of wounds or for the reduction of inflammatory response.
